(19) United States
(12) Patent Application Publication
(10) Pub. No.: US 2013/0052735 A1
(43) Pub. Date: Feb. 28, 2013

(54) Microporous Thermoplastic Sheets

(71) Applicants: Michael Edward DeRosa, Painted Post, NY (US); Todd Michael Upton, Eliot, ME (US); Ying Zhang, Corning, NY (US)

(72) Inventors: Michael Edward DeRosa, Painted Post, NY (US); Todd Michael Upton, Eliot, ME (US); Ying Zhang, Corning, NY (US)

(21) Appl. No.: 13/217,818

(22) Filed: Aug. 25, 2011

(51) Int. Cl. C12N 5/071; C08F 112/08; C08F 222/08; C08J 9/28

(52) U.S. Cl. 435/396; 521/64

(57) ABSTRACT

Thermoplastic polymeric sheets are rendered microporous and remain substantially flat by contacting the sheet with a first fluid composition that contains or more solvents for the polymeric sheet to render the sheet microporous and then contacting the microporous sheet with a second fluid composition that is substantially free of solvents for the polymer and that contains a non-solvent that is miscible with the one or more solvents of the first composition. Contacting the microporous sheet with the second fluid composition preferably occurs prior to substantial evaporation of the first fluid compositions, or solvents thereof, from the microporous sheet.

### FIELD

The present disclosure relates to, among other things, microporous thermoplastic sheets and methods for making such sheets, and to cell culture article containing such sheets.

### BACKGROUND

Microporous polymeric sheets have been used for a variety of purposes, including microfluidic reaction chambers, filtering devices, and three-dimensional substrates for cell culture. Such sheets have been made via a variety of processes. For example, solution-based casting methods have been used for many years and have been described in detail. Though there are many variations, a common process used employs a polymer phase separation process. In such processes, the polymer is typically dissolved in a solvent, the dissolved polymer is cast, and solvent is partially evaporated to produce a plasticized or gelled polymer, which is then immersed in a bath of non-solvent, which is miscible with the solvent. When the gelled polymer contacts the non-solvent bath, the polymer precipitates by phase separation forming a porous polymeric material. Other methods for making microporous sheets for purposes of three dimension cell culture substrates include emulsion polymerization and assembling three dimensional woven polymer grid structures. However, such processes can be time consuming or expensive to perform.

### BRIEF SUMMARY

The present disclosure describes, among other things, a method for fabricating a microporous thermoplastic polymeric sheet from a relatively non-porous thermoplastic sheet having a birefringence of 0.0001 or greater. The non-porous sheet is first contacted with a fluid composition comprising a solvent for the polymer to generate a microporous surface on the polymeric sheet and is then contacted with a second fluid composition that is substantially free of solvents for the polymer but which includes a non-solvent miscible with the solvent of the first fluid composition to extract the solvent from the microporous sheet. It has been found that microporous thermoplastic sheets made according to such a process can be made substantially flat, in contrast to similar processes in which the solvent from the first fluid composition is allowed to evaporate.

In embodiments, a method for fabricating a substantially flat microporous thermoplastic polymeric sheet is described herein. The method includes (i) providing a thermoplastic polymeric sheet having a birefringence of 0.0001 or greater and a thickness of less than 500 micrometers; and (ii) contacting the sheet with first composition having one or more solvents for the polymeric sheet to generate a microporous surface on the sheet. The first composition has Hansen relative energy difference from the thermoplastic polymer of between 0.5 and 2. The method also includes contacting the sheet having the microporous surface with a second composition that is substantially free of solvents for the sheet having the microporous surface. The second composition comprises a non-solvent that is miscible with the one or more solvents in the first composition to extract the one or more solvents in the first composition from the sheet having the microporous surface. The method further includes drying the sheet having the microporous surface to remove the second composition thereby producing the substantially flat microporous thermoplastic polymeric sheet.

Cell culture article fabricated using such flat microporous thermoplastic polymeric sheets are also described herein.

The devices, articles and methods described herein may provide one or more advantages over prior thermoplastic sheets having microporous structure or methods for making such sheets. For example, embodiments of the methods described herein allow for generation of large microporous polymer sheets that are substantially flat, which may not be achievable when solvent is allowed to evaporate. In addition, by contacting the microporous sheet, which has been contacted with a solvent to render the sheet microporous, with a non-solvent to extract the solvent, more uniform pore thickness control is achievable than methods in which the solvent is evaporated. Further, embodiments of processes described herein allow for production of flat microporous sheets useful as inserts for cell culture at a cost that is greatly reduced relative to existing procedures for making similar inserts. Additionally, embodiments of cell culture inserts described herein provide for more suitable cell culture surfaces relative to previously available inserts. These and other advantages of the various embodiments of the devices and methods described herein will be readily apparent to those of skill in the art upon reading the disclosure presented herein.

### BRIEF DESCRIPTION OF THE DRAWINGS

FIG. 1 is a schematic diagram of a Hansen solubility sphere of a polymer and shows representative coordinates of a test solvent or mixture.

FIG. 2 is a schematic diagram of a Hansen solubility sphere showing coordinates relative to the sphere of solvents capable of causing micropore formation.

FIG. 3 is a schematic diagram showing a substantially flat microporous sheet.

FIG. 4 is a schematic diagram showing various steps for forming a substantially flat microporous sheet from a thermoplastic polymer sheet.

FIG. 5 is a schematic diagram showing various steps for generating inserts for a multi-well cell culture plate from a substantially flat microporous sheet.

FIG. 6 is a two dimensional Hansen solubility parameter plot (δ_{P} vs. δ_{H}) of solvents, non-solvents and solvent/non-solvent mixtures for polystyrene.

FIG. 7 is an image of a substantially flat microporous sheet made according to a two-step solvent process (A) and from a single step solvent process (B) as described in the EXAMPLES herein.

FIG. 8 is an image of inserts for a multi-well cell culture plate produced from a substantially flat microporous (see insert) sheet.

FIG. 9 is an image of inserts for a multi-well cell culture plate produced from a substantially flat microporous sheet made according to a two-step solvent process (A) and from a single step solvent process (B) as described in the EXAMPLES herein.

FIG. 10 is a bar graph showing levels of expression of various genes from hepatocytes cultured on a variety of surfaces, including inserts produced from a substantially flat microporous sheets as described herein.

The schematic drawings presented herein are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to.” It will be understood that the terms “consisting of” and “consisting essentially of” are subsumed in the term “comprising,” and the like. For example, a method for fabricating microporous surface on a polystyrene article that comprises (i) providing a polystyrene article having a birefringence of 0.0001 or greater; (ii) contacting a surface of the article with a composition comprising a solvent for the polystyrene, wherein the composition is configured to cause swelling of the polystyrene article without dissolving the polystyrene article; and (iii) removing the composition from the polystyrene article may consist of, or consist essentially of, providing the article, contacting the surface of the article with the composition and removing the composition.

“Consisting essentially of”, as it relates to a compositions, articles, systems, apparatuses or methods, means that the compositions, articles, systems, apparatuses or methods include only the recited components or steps of the compositions, articles, systems, apparatuses or methods and, optionally, other components or steps that do not materially affect the basic and novel properties of the compositions, articles, systems, apparatuses or methods.

Any direction referred to herein, such as “top,” “bottom,” “left,” “right,” “upper,” “lower,” “above,” below,” and other directions and orientations are described herein for clarity in reference to the figures and are not to be limiting of an actual device or system or use of the device or system. Many of the devices, articles or systems described herein may be used in a number of directions and orientations.

As used herein, “microporous structure” refers to a structure having pores or interstices of an average diametric size of less than 1000 micrometers.

As used herein, “pore” means a cavity or void in a surface, a body, or both a surface and a body of a solid article, where the cavity or void has at least one outer opening at a surface of the article.

As used herein, “interstice” means a cavity or void in a body of a solid polymer not having a direct outer opening at a surface of the article, i.e., not a pore, but may have an indirect outer opening or pathway to an outer surface of the article by way of one or more links or connections to adjacent or neighbor “pores” “interstices,” or a combination thereof.

As used herein, a “solvent” for a polymeric sheet is a composition capable of causing swelling or solubilization of at least a portion of the polymeric sheet when contacted with the sheet; e.g. at room temperature. A “good” solvent is a solvent that solubilizes the polymeric sheet. A “non-solvent” for a polymeric sheet means a composition that does not cause swelling or solubilization of the polymeric sheet when contacted with the sheet.

As used herein, a “polymeric sheet,” or the like, means a polymeric article having length, width and thickness dimensions. Wherein the length and width are substantially greater than the thickness. A sheet is may be generally rectangular, but may be of any other general shape such as circular or irregular. A polymeric sheet may be a film, membrane or the like. In embodiments, the polymeric sheet has a thickness of 500 micrometers or less.

As used herein, “substantially flat,” in the context of polymeric sheet, means a sheet that is substantially free of wrinkles, curls or distortions. In embodiments, more than 70% of a surface of a “substantially flat” sheet contacts a flat surface when placed horizontally on the flat surface, absent external forces. In embodiments, more than 80%, or more than 90%, of the surface of a substantially flat sheet contacts a flat surface when placed horizontally on the flat surface.

As used herein, “substantially free of solvents for a polymer,” in the context of a composition, means that the composition may contain some amount of one or more solvents for the polymers so long as the overall composition does not act as a solvent for the polymer. In embodiments, a composition that is substantially free of solvents for a polymer contains less than 5% by volume of a solvent for the polymer; e.g., less than 1% or less than 0.5%.

As used herein, “drying,” in the context of a polymeric sheet, means the process of evaporating, or otherwise removing, substantially all of a liquid composition from the sheet. By way of example, a polymeric sheet may be considered to be dry if the sheet is within 5% of its dry weight (e.g., weighs between 100% and 105% of its dry weight).

As used herein, “substantially distort, wrinkle or curl,” in the context of a polymeric sheet, means that a process or act that causes a substantially flat sheet to no longer be substantially flat.

As used herein, a “non-porous” sheet is a sheet that is substantially free macroporosity. Macroporosity, as used herein, means having pores of a size greater than 500 nanometers. Substantially free of macroporosity, as used herein, means that less than 5% of the surface of a sheet has pores that are greater than 500 nanometers.

The present disclosure describes, among other things, methods for forming substantially flat microporous polymeric sheets. A non-porous polymeric sheet is rendered porous via contact with a first composition having one or more solvents for the polymer. The first composition has a solubility strength sufficient to swell the polymer but not dissolve the polymer. A microporous structure is formed on the sheet during contact with the first composition. It has been found that the pore formation process can be arrested by contacting the microporous sheet with a second fluid composition that does not contain a solvent for the polymer but contains one or more non-solvents that are miscible with the one or more solvents of the first fluid composition. In contrast with processes in which the first composition, or components thereof, is allowed to evaporate from the microporous sheet, processes as described herein in which the microporous sheet is contacted with the second fluid composition to extract the one or more solvents from the sheet, the microporous sheet can be made to be substantially flat.

1. Formation of Thermoplastic Sheet

It has been found that, in order to produce a microporous surface from a thermoformed thermoplastic article, such as a thermoplastic sheet, the article should be made with at least a minimum amount of molecular orientation. Without intending to be bound by theory, it is believed that this will allow the solvent to penetrate into the surface and create the microporous texture. The relative level of molecular orientation of a transparent thermoplastic can be determined by the degree of optical birefringence. The birefringence (Δn) is defined as

Δn=n₁−n₂

where n₁ an n₂ are the refractive indices of light polarized parallel (n₁) and perpendicular (n₂) to the deformation or flow direction of the polymer during the forming process. When an oriented transparent polymer article is placed between crossed polarizing sheets, the birefringence pattern can be seen with multiple colored fringes. The birefringence can be calculated from these fringes. Higher order fringes indicate higher level of birefringence and hence orientation.

The residual stress in a material is also related to the birefringence of the material as well. The birefringence is related to the stress (σ) by a constant called the stress optic coefficient (SOC) as

$$S O C = \frac{Δ n}{σ} .$$

To achieve high birefringence, and hence molecular orientation, a polymer can be formed with high residual stress by controlling the molding or extruding parameters such as injection speed, melt temperature, gate location and size, mold temperature, etc.

For example, it has been found that the size and location of the injection gate into a mold cavity may be chosen to create a sufficiently high shear zone across the volume of the mold to increase molecular orientation. Similarly, increased injection speeds tend to result in increased shear stress and thus increased molecular orientation. By way of further example, the further below the glass transition temperature of a particular polymeric material that the setting of the mold temperature is held, the more the residual stress that may be molded into the article. For sheets or films, uni- or biaxial stretching results in molecular orientation. It will be understood that these are merely examples of how one can produce an article having sufficient molecular orientation to achieve a birefringence of greater than 0.0001. Other techniques may be used and are generally known in the art, including drawing, calendaring, blow molding, film blowing and the like.

Thermoplastic sheets having sufficient residual stress or birefringence may be formed by any suitable method, such as extrusion, blow molding, injection molding or the like. The articles for use with the methods described herein preferably have a birefringence 0.0001 or greater, such as 0.001 or greater or 0.01 or greater.

The methods described herein may be applied to any suitable thermoplastic polymer, including glassy amorphous thermoplastic polymers such as polystyrenes, polycarbonates, polymethylmethacrylate or other acrylic polymers, cyclic olefin copolymers, styrene maleic anhydride polymers, or blends thereof. The articles made from these polymers may be sheets or the like.

In embodiments, a thermoplastic sheet that is rendered microporous according to the teachings presented herein has a thickness of 500 micrometers or less, such as 250 micrometers or less or 100 micrometers or less.

2. Formation Microporous Structure

In embodiments, at least a portion of a non-porous polymeric sheet is rendered microporous by contacting the polymeric sheet with a fluid composition comprising one or more solvent for the polymer. The solubility strength of the fluid composition is configured to cause swelling of the polymeric sheet, but not to dissolve the polymer; e.g. at room temperature. The solvent strength of the fluid composition may be controlled by including more than one solvent in the fluid composition or by including one or more non-solvents in the composition. Preferably, all solvents or non-solvents included in the fluid composition are miscible. If a fluid composition configured to render a non-porous sheet porous includes a good solvent, the composition will generally also include a non-solvent to properly adjust the solubility strength of the composition.

The solubility strength required to induce micropore formation will depend on the nature of the polymer employed and the residual stress in the polymeric sheet, with a lower solvent strength needed when the sheet has higher residual stress or birefringence. A solvent strength of a solvent or solvent mixture that is suitable for inducing pore formation on a polymeric sheet may be determined using Hansen solubility parameters (see, e.g., Hansen, C. M., Hansen Solubility Parameters a User's Handbook 2nd Ed., CRC Press, Boca Raton, 2007). According to Hansen, the total cohesion energy (E) of a liquid is defined by the energy required to convert a liquid to a gas. This can be experimentally measured by the heat of vaporization. Hansen described the total cohesion energy as being comprised of three primary intermolecular forces: atomic dispersion forces (E_{D}), molecular permanent dipole-dipole interactions (E_{P}), and molecular hydrogen bonding interactions (E_{H}). When the cohesion energy is divided by the molar volume (V) the total cohesive energy density of the liquid is given by

E/V=E_{D}/V+E_{P}/V+E_{H}/V. (1)

The solubility parameter (δ) of the liquid is related to the cohesive energy density by

δ=(E/V)^{1/2} (2)

where δ is the Hildebrand solubility parameter. The three Hansen solubility components of a liquid are thus given by

δ²=δ_{D}²+δ_{P}²+δ_{H}². (3)

These three parameters have been tabulated for thousands of solvents and can be used to describe polymer-solvent interactions (see, e.g., Hansen, 2007).

Solubility parameters exist for solid polymers as well as liquid solvents (see, e.g., Hansen, 2007). Polymer-solvent interactions are determined by comparing the Hansen solubility parameters of the polymer to that of a solvent or solvent mixture defined by the term Rₐ as

R^{a}=4(δ_{D2}−δ_{D1})²+δ_{P2}−δ_{P1})²+(δ_{H2}−δ_{H1})² (4)

where subscripts 1 and 2 refer to the solvent or solvent mixture and polymer respectively. Rₐ is the distance in three dimensional space between the Hansen solubility parameters of a polymer and that of a solvent. A “good” solvent for a particular polymer has a small value of Rₐ. This means the solubility parameters of the polymer and solvent are closely matched and the solvent will quickly dissolve the polymer. Rₐ will increase as a solvent's Hansen solubility parameters become more dissimilar to that of the polymer.

The solubility of a particular polymer is not technically described by just the three parameters in Equation (3). A good solvent does not have to have parameters that perfectly match that of the polymer. There is a range of solvents that will work to dissolve the polymer. The Hansen solubility parameters of a polymer are defined by δ_{D}, δ_{P}, and δ_{H} which are the coordinates of the center of a solubility sphere which has a radius (Rₒ). Rₒ defines the maximum distance from the center of the sphere that a solvent can be and still dissolve the polymer.

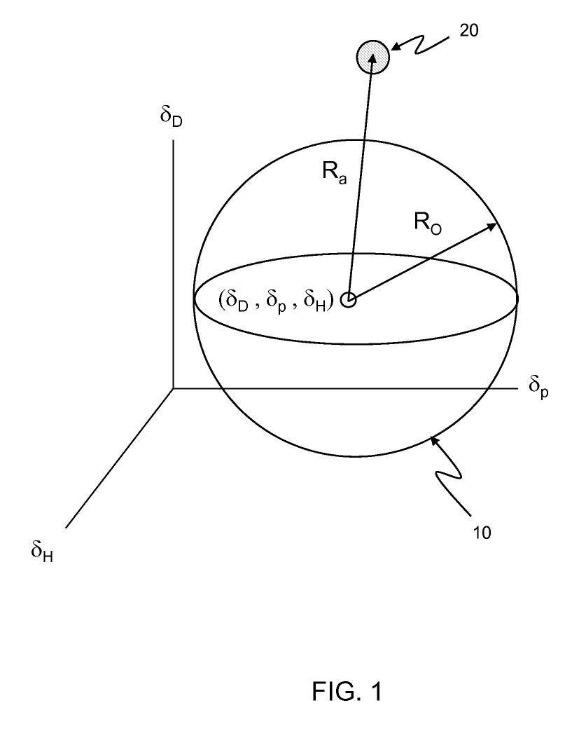

A schematic of a polymer solubility sphere 10 and a test solvent or mixture coordinates 20 are shown in FIG. 1, where the sphere 10 defined by its center coordinates (δ_{D}, δ_{P}, δ_{H}) and a radius Rₒ. Solvents that lie within the sphere 10 will dissolve the polymer. The coordinates 20 of an example of a test solvent or solvent mixture, which are at a distance, Rₐ, from the center of the solubility sphere, are also depicted in FIG. 1.

The strength of a solvent for a polymer is determined by comparing Rₐ to Rₒ. A term called the Relative Energy Difference (RED) is given by

RED=Rₐ/Rₒ. (5)

Using RED values is a simple way to evaluate how “good” a solvent will be for a given polymer. Solvents or solvent mixtures that have a RED number much less than 1 will have Hansen solubility parameters close to that of the polymer and will dissolve the polymer quickly and easily. Liquids that have RED numbers much greater than 1 will have Hansen solubility parameters further away from the polymer and will have little or no effect on the polymer. Liquids that have RED numbers close to one will be on the boundary between good and poor solvents. These liquids usually swell the polymer and belong to a class of solvents that typically cause environmental stress cracking and crazing (see, e.g., Hansen, C. M.; Just, L., “Prediction of Environmental Stress Cracking in Plastics with Hansen Solubility Parameters, Ind. Eng. Chem. Res., 40, 21-25, 2001).

We have found that solvent or solvent mixtures that have RED values in a range of the polymer solubility boundary have been found to cause microporous formation on molded thermoplastic articles. In particular, fluid compositions comprising one or more solvents, which may also contain one or more non-solvents, that have a RED of between about 0.5 and about 2 may be suitable for forming microporous structures on polymeric articles. Preferably, the fluid composition has a RED of between about 0.75 and about 1.5, such as between about 0.8 and about 1.4 or between about 0.9 and about 1.2.

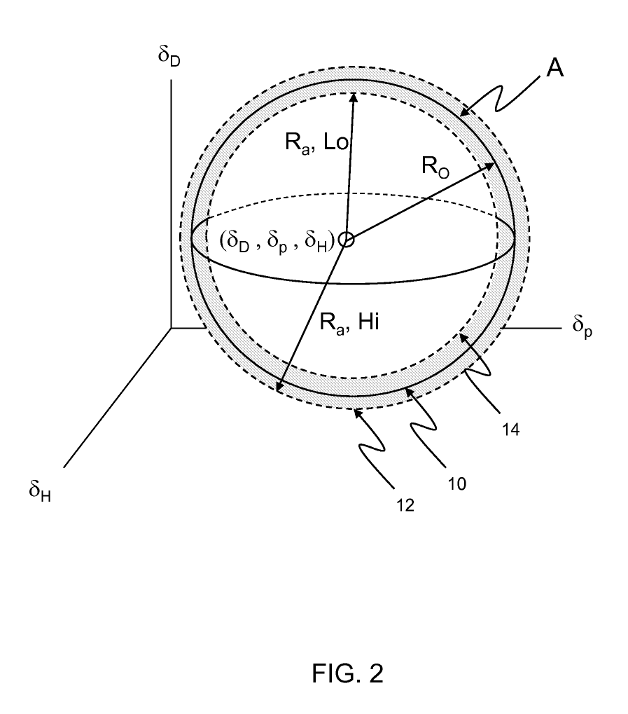

FIG. 2 is a schematic of illustration fluid compositions having suitable solubility parameters to form microporous surfaces. The polymer solubility sphere is defined by its center coordinates (δ_{D}, δ_{P}, δ_{H}) and a radius Rₒ. Solvent and solvent mixtures that will form microporous surfaces will have solubility parameters that reside in a range around the polymer solubility sphere, as indicated by the shaded area. The outer boundary 12 of solubility parameters that are suitable for forming the microporous surfaces is depicted in FIG. 2 as being defined by the radius Rₐ, Hi and defines the upper RED value. The lower boundary 14 of solubility parameters that are suitable for forming the microporous surfaces is depicted in FIG. 2 as being defined by the radius Rₐ, Lo and defines the lower RED value. A similar framework for environmental stress crazing was discussed by Hansen (see, Hansen, 2007 and Hansen, 2001). Hansen used this for the purpose of describing mechanical reliability of polymers and treated stress crazing as phenomenon to be avoided. Here we are using this range of polymer/solvent interactions to define the desirable characteristics for producing microporous surfaces.

It will be understood that the width of this RED value range depends on the amount of residual stress in the polymer article, with higher residual stress resulting in higher RED values. That is, the higher the amount of residual stress, or birefringence, the larger the RED value will be for the upper boundary. Polymeric articles that have lower stress or birefringence will require solvents or solvent mixtures that are closer to the center of the sphere within the shaded region to produce porous surfaces.

It will also be understood that the values of R₀ value of a given polymer may change depending on the amount of residual stress or birefringence of the article. The value obtained for Rₒ may also change based on the solvents or non-solvents used to determine the R₀ value. If solvents or combinations of solvents and non-solvents are used that are within the micropore forming range (e.g. shaded area of the sphere in FIG. 2), then the value of R₀ may more readily change depending on residual stress or birefringence. However, if solvents or combinations of solvents and non-solvents are used that are not within the micropore forming range, the determined R₀ value may not change with changing residual stress or birefringence values.

The fluid composition comprising the solvent may include one or more solvents and may optionally include one or more non-solvents. The one or more solvents or non-solvents may be selected based on the Hansen solubility parameters, e.g. as described above. Any solvent suitable for solubilizing or swelling a polymer of the article may be employed. Such solvents are generally known in the art. For example, for polystyrene, suitable solvents include tetrahydro furan, methylethyl ketone, ethyl acetate, and acetone. For cyclic polyolefins suitable solvents include methylene chloride, and tetrahydrofuran. For styrene maleic anhydride polymeric sheets, suitable solvents include acetone, tetrahydrofuran, 1,3-dioxolane, methylethyl ketone, toluene, ethyl acetate, and N-methylpyrolidone. It will be understood that these are only a few examples of the suitable solvents that may be used for these polymers and that other solvents may readily be used and that other polymers with appropriate solvents may be used in accordance with the teachings herein to generate a microporous structure.

Any one or more non-solvents may be employed. As with solvents, some non-solvents may be selective to the polymeric article for which it is desirable to impart a microporous region. However, many non-solvents will work with most, if not all, polymers. By way of example, suitable non-solvents for polystyrene include water and an alcohol, such as a C1-C4 unsubstituted alcohol, which includes isopropanol, ethanol, and methanol. For cyclic polyolefins, suitable non-solvents include water and an alcohol, such as a C1-C4 unsubstituted alcohol. For styrene maleic anhydride polymers, suitable non-solvents include water and C1-C4 unsubstituted alcohols which include isopropanol, ethanol, and methanol. It will be understood that these are only a few examples of the suitable non-solvents that may be used for these polymers and that other non-solvents may readily be used and that other polymers with appropriate non-solvents may be used in accordance with the teachings herein to generate a microporous structure.

As indicated above, it has been found that the solubility strength of the composition comprising the solvent or solvent mixture should be finely controlled to produce a desired microporous structure. It will be understood that the ratio and composition of solvent and non-solvent will vary depending on a number of factors, including the composition of the polymeric article and the solubility of the polymeric article in the solvent employed. In some cases, no non-solvent is required to achieve a desired solubility parameter. In other cases, the non-solvent constitutes up to 70 percent or more of the volume of the composition comprising the one or more solvents.

By way of example, it has been found that solvent compositions having the following ratios, on a volume/volume basis, of solvent and non-solvent are suitable for forming microporous structures from polystyrene articles having a birefringence of 0.0001 or greater: tetrahydrofuran (THF)/isopropanol in range of 35/65-50/50; THF/ethanol in a range of 35/65-50/50; ethyl acetate/isopropanol in a range of 45/55-65/35; and THF/water in a range of 40/60-70/30, such as 45/55-65/35. By way of further example, it has been found that solvent compositions having the following ratios, on a volume/volume basis, of solvent and non-solvent are suitable for forming microporous structures from cyclic olefin copolymer articles having a birefringence of 0.0001 or greater: methylene chloride (single solvent); THF/isopropanol in a range of 75/25-90/10; and THF/water in a range of 80/20-98/2, such as 90/10-95/5. It will be understood that these are just examples that were found to work successfully and these do not constitute an exhaustive list of solvents, non-solvents, and polymers for which the processes described herein will produce microporous surface structures.

To the extent that the ranges of ratios of solvent and non-solvent may vary from polymeric article to polymeric article and from solvent to solvent; a suitable range may be readily identified by those of skill in the art. For example, (i) one may try a variety of ratios of known solvents and non-solvents for a particular polymer to determine whether the ratio is suitable for forming a porous structure from the article, (ii) identify those ratios that are suitable and expand around those ratios to find the boundaries of suitable ranges. Any suitable test or assay may be employed to determine whether the composition comprising solvent and non-solvent is capable of imparting a microporous structure to at least portion of the polymeric article may be performed. For example, microscopic examination of article after contact and removal of the solvent/non-solvent composition may be used to identify whether suitable porous regions have formed. Of course, Hansen solubility parameters may be employed to determine which one or more solvents or non-solvents, and their appropriate amounts, may be used to generate a microporous surface on a polymeric article, such as a polymeric sheet.

The depth that the generated microporous structure may extend through the article may vary and may be controlled by controlling solvent contact time, temperature, and the like. For example, the microporous region may be formed only on the surface of the article, having a depth of about, e.g., 10 micrometers to about 100 micrometers, or may extend through the entire depth of the article, depending on the conditions used. The thickness of the non-porous starting article will also affect the extent to which the microporous network extends through the article.

The non-porous starting thermoplastic article may be contacted with the fluid composition that includes a solvent in any suitable manner. For example, the article may be submersed into the fluid composition, the composition may be sprayed on, pipetted on, casted on, inkjetted on, contacted printed on, dropped on, or otherwise applied to the article, the composition maybe vaporized and applied to the article, and the like. It has been found that dipping the article into the liquid composition serves as a convenient and readily accessible method for contacting the article with the composition. It has also been found that microporous structures can readily be generated from the articles at room temperatures, further adding to the convenience. Of course, the temperature may be varied as desired or practicable to achieve a suitable microporous network.

The pore size of the resulting microporous structure may vary depending on, among other things, the composition of the polymeric material, the birefringence of the material, the solvent and non-solvent used, and the like. It has been found that the average size of the pores generated can be moderately controlled by the solvent composition employed. Average pore sizes generated using the methods described herein, in some embodiments, can range from between 1 micrometer to 500 micrometers, such as between 10 and 200 micrometers. While the mechanism of pore formation is not entirely understood, using an alcohol (e.g. isopropanol or ethanol) as a nonsolvent tends to favor the formation of smaller average pore sizes, and water as a nonsolvent tends to favor formation of larger pore sizes on polystyrene substrates.

In addition, the resulting microporous structure that forms from the polymeric article may be an interconnected open cell structure or a non-interconnected open cell structure. Again, while the mechanism is not entirely understood, we have found that higher degrees of orientation (higher birefringence) tends to favor formation of more highly interconnected porous structures. Microscopic examination of the microporous structure may give an indication as to whether the resulting microporous structure is interconnected or non-interconnected. By way of further example, one may employ a liquid wicking test to determine whether the generated porous network is interconnected. If a liquid is blocked from moving across the surface of the microporous structure and is capable of moving though the generated porous network, then the generated porous network is interconnected and has an open cell configuration. Any suitable liquid wicking test may be employed. By way of example, such a test may be performed generally as described in EXAMPLE 5 of copending patent application No. 61/377,549, filed on the same day as the present application, entitled FLEXIBLE MICROFLUIDIC DEVICE WITH INTERCONNECTED POROUS NETWORK, naming Po Ki Yuen and Michael E. DeRosa as inventors, and having attorney docket no. SP10-234P, which application is hereby incorporated herein by reference in its entirety to the extent that it does not conflict with the present disclosure.

In various embodiments, a polymeric article with a patterned microporous structure is fabricated. To produce the patterned article, a mask may be applied to a surface of the article prior to contacting the article with the composition comprising the one or more solvents. Any suitable mask may be used. The mask should prevent the surface of the article from being contacted with the solvent composition, e.g., when submersed in the composition. Additionally, the mask should be readily removable from the article and should not be soluble in the one or more solvents used. In many embodiments, self adhesive tape or other films may be used as a mask. In some embodiments, it may be desirable to mask one entire surface of the article and to pattern mask the opposing surface to produce a desired microporous structure only one surface of the article.

One convenient way to form a film mask with a desired pattern is to use a desktop digital cutting device, such as described in, for example, P. K. Yuen and V. N. Goral, “Low-cost rapid prototyping of flexible microfluidic devices using a desktop digital craft cutter”, Lab on a Chip, 2010, 10, 384-387. Of course, any other suitable method may be used to cut or produce a mask to a desired pattern.

After the pores are formed on the polymeric sheet by the fluid composition that includes the solvent or solvent mixture (whether patterned or continuous), the porous sheet is contacted with a second composition that contains one or more non-solvents that are miscible with the one or more solvents in the pore forming composition. The porous sheet may be contacted with the second non-solvent composition in any suitable manner. For example, the porous sheet may be dipped or immersed in a bath of the second composition, the second composition may sprayed or poured onto the porous sheet, or the like.

To maintain flatness of the sheet, the porous sheet is contacted with the second non-solvent composition while the porous sheet is still swollen with the first solvent composition. It has been found that if a substantial amount of the first solvent composition, or components thereof, evaporate from the porous sheet before the sheet is contacted with the second solvent, the sheet tends to wrinkle, curl or buckle. Thus, in embodiments, the porous sheet is contacted with the porous sheet the sheet begins to substantially wrinkle, curl or distort due to evaporation of the first solvent composition. It will be understood that the time that the sheet begins to substantially wrinkle, curl or distort due to evaporation of the first solvent composition will vary depending on the volatility of the first solvent composition, or components thereof, under the conditions employed.

In embodiments, the porous sheet is contacted with the second non-solvent composition as soon as practicable after removal from the first solvent composition (or ceasing of contact with the first solvent composition). In embodiments, the porous sheet is contacted with the second non-solvent composition within 60 seconds of removal from the first solvent composition (or ceasing of contact with the first solvent composition), such as within 30 seconds, within 10 seconds, or within 5 seconds.

The second composition is preferably configured to arrest the pore forming process, extract the first composition, or components thereof, from the pores of the porous sheet, and render the sheet sufficiently rigid to handle. By contacting the porous sheet with the second non-solvent composition (as opposed to drying the first composition from the sheet), pore formation may be more reproducibly arrested, resulting in more uniform pore generation from polymeric sheet to polymeric sheet.

The second composition may include any suitable non-solvent (e.g., those non-solvents described above). Preferably the first solvent composition is miscible in the second non-solvent composition. In embodiments, the second composition has a Hansen RED of greater than 2, such as 3 or greater.

The second non-solvent composition may be removed from the polymeric sheet in any suitable manner, such as removing the sheet from the second composition source and drying. Drying may be facilitated by increasing temperature, suction, vacuum stripping, or blowing air or nitrogen, or the like. It has been found that evaporating the second non-solvent composition from the porous sheet results in a porous sheet that substantially retains its flatness without substantial wrinkling, curling or distortion, in contrast to evaporating the first solvent composition from the sheet.

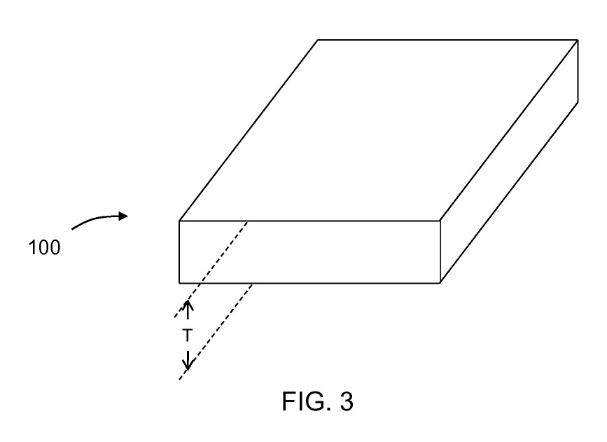

Referring now to FIG. 3, a schematic drawing of a polymeric sheet 100 is shown. The depicted polymeric sheet 100 is substantially non-porous and flat and has a thickness, T. Any suitable polymeric sheet 100 may be subjected to the processes described herein. As discussed above, the polymeric sheet 100 may be a thermoplastic polymeric sheet, at least a portion of which has a birefringence of 0.0001 or greater. The sheet 100 may have any suitable thickness, T, such as 500 micrometers or less or 100 micrometers or less.

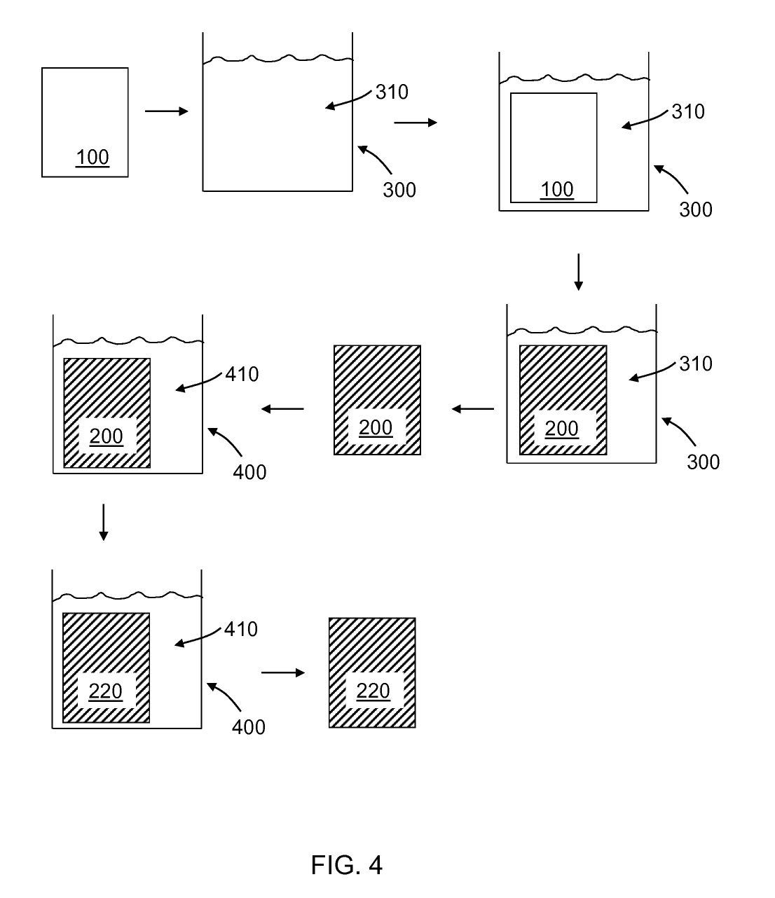

Referring now to FIG. 4, a schematic diagram of a process for rendering a polymeric sheet 100 porous (or more porous) is shown. The starting polymeric sheet 100 may be immersed in a bath 300 of a first solvent composition 310, which contains one or more solvents and, optionally, one or more non-solvents, as described above. Upon contact of the sheet 100 with the first solvent composition, the sheet, or one or more surfaces thereof (depending on masking, etc.), becomes porous, thereby producing a microporous polymeric sheet 200. The microporous sheet 200 is removed from the first bath 300 and at least a portion of the sheet 200, or surface thereof, is swollen with the first solvent composition and may be in a softened or plasticized state. The microporous sheet 200 having at least a portion swelled or plasticized with (or by) the first fluid composition is then contacted with a second non-solvent composition, e.g. as discussed above.

In FIG. 4, the porous polymeric sheet 200 is immersed in a bath 400 containing the second non-solvent composition 410, which may include one or more non-solvents miscible with the first solvent composition or components thereof. Immersing the porous sheet 200 in the second composition 410 arrests the pore forming process and extracts the first solvent from the sheet, rendering a microporous sheet 220 that is sufficiently rigid from routine handling. The sheet 220 may be dried and the second composition evaporated so the sheet 220 may be packaged or used for its intended purpose.

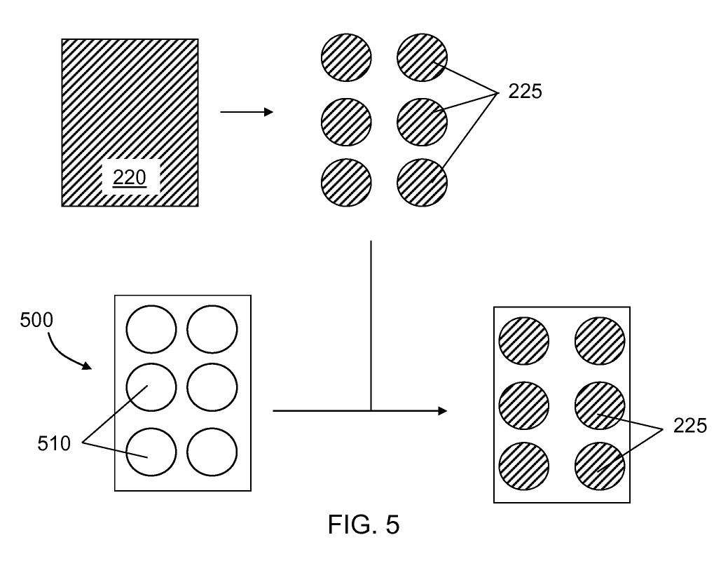

As shown in FIG. 5, the polymeric sheet 220 may be cut, punched, or the like to produce inserts 225 for lining the bottom of wells 510 of cell culture plates 500. The inserts 225 may be used to provide a three-dimension surface for cell culture. The inserts 225 be simply disposed in the well 510 or may be adhered or otherwise affixed to the well 510.

The polymeric sheets 220, or portions thereof, may be used for any suitable purpose, such as filtration, microfluidics, lateral flow assays, or the like.

3. Modification of Properties of Microporous Network

Many polymeric sheets have hydrophobic surfaces, and rendering the surfaces microporous may increase the hydrophobicity of the article. In some applications of the microporous sheets, the increased hydrophobicity may be desirable. However, in other applications, a more hydrophilic surface may be desired. The microporous surfaces generated according to the methods described above may be treated in any suitable manner to increase the hydrophilicity or wettability of the surface. For example, plasma treatment, such as oxygen plasma treatment, may be employed. One suitable method for forming more hydrophilic surface that may be employed is Corning Incorporated's CELLBIND process, e.g. as described in U.S. Pat. No. 6,617,152, which is incorporated herein by reference in its entirety to the extent that it does not conflict with the present disclosure. Other methods for increasing hydropilicity or wettability of a surface, such as those described in U.S. Pat. No. 4,413,074, which is incorporated herein by reference in its entirety to the extent that it does not conflict with the present disclosure, may be employed. In U.S. Pat. No. 4,413,074 a hydrophobic polymer surface is contacted with a solution containing hydroxyalkyl cellulose and a perfluorocarbon surfactant in water (or a mixture of water and one or more aliphatic alcohols) to form a layer of the solution on the surface. The surface is then heated to form a bond between the cellulose and the surface, rendering the surface more hydrophilic. Of course, any other methods such as UV ozone or arc plasma may be employed to increase the hydrophilicity or wettability of a microporous surface.

In some embodiments, a hydrophobic thermoplastic sheet having a microporous structure is rendered hydrophilic in a patterned manner. To produce such an sheet having patterned hydrophilic regions, a mask may be applied to a surface of the sheet prior to subjecting the microporous structure of the sheet to the hydrophilic treatment. Any suitable mask may be used. In many cases, the mask may be a mask as described above with regard to producing a patterned microporous structure. For example, the mask may be formed from self adhesive tape or other film. Regardless of composition of the mask, the mask should prevent the underlying surface of the article from being rendered hydrophilic when the sheet is subjected to the hydrophilic treatment. Preferably, the mask is readily removable from the sheet following the treatment.

4. Summary of Selected Disclosed Aspects

This disclosure in various aspects describes methods and articles.

In a first aspect, method for fabricating a substantially flat microporous thermoplastic polymeric sheet, comprises (i) providing a thermoplastic polymeric sheet having a birefringence of 0.0001 or greater and a thickness of less than 500 micrometers; (ii) contacting the sheet with first composition having one or more solvents for the polymeric sheet, wherein the first composition has Hansen relative energy difference from the thermoplastic polymer of between 0.5 and 2 to generate a microporous surface on the sheet; (iii) contacting the sheet having the microporous surface with a second composition that is substantially free of solvents for the sheet having the microporous surface, wherein the second composition comprises a non-solvent that is miscible with the one or more solvents in the first composition to extract the one or more solvents in the first composition from the sheet having the microporous surface; and (iv) drying the sheet having the microporous surface to remove the second composition thereby producing the substantially flat microporous thermoplastic polymeric sheet.

A second aspect is a method of the first aspect, wherein the first composition has Hansen relative energy difference from the polymer of between 0.75 and 1.6.

A third aspect is a method of the first aspect, wherein the first composition has Hansen relative energy difference from the polymer of between 0.8 and 1.5.

A fourth aspect is a method of the first aspect, wherein the first composition has Hansen relative energy difference from the polymer of between 0.9 and 1.4.

A fifth aspect is a method of any of the first four aspects, wherein the first composition comprises one or more non-solvents for the polymeric sheet.

A sixth aspect is a method of any of the first five aspects, wherein the thermoplastic polymeric sheet is a polystyrene sheet.

A seventh aspect is a method of the sixth aspect, wherein the first composition comprises a solvent selected from the group consisting of tetrahydrofuran, ethylacetate, toluene, acetone, and 1,3 dioxolane, and a non-solvent selected from the group consisting of water, isopropanol, ethanol, propylene carbonate, and dimethyl sulfoxide.

An eighth aspect is a method of the sixth aspect, wherein the first composition comprises a solvent and non-solvent mixture selected from the group consisting of (i) tetrahydrofuran and water, wherein the respective volume percentages are between 50/50 and 65/35; (ii) tetrahydrofuran and isopropanol, wherein the respective volume percentages are between 35/65 and 45/55; (iii) tetrahydrofuran and propylene carbonate, wherein the respective volume percentages are between 37/63 and 50/50; (iv) ethylacetate and isopropanol, wherein the respective volume percentages are between 60/40 and 70/30; (v) toulene and dimethyl sulfoxide, wherein the respective volume percentages are between 25/75 and 30/70; acetone and isopropanol, wherein the respective volume percentages are between 70/30 and 80/20; and (vi) 1,3 dioxolane and water, wherein the respective volume percentages are between 60/40 and 80/20.

A ninth aspect is a method of any of the first five aspects, wherein the thermoplastic polymeric sheet is a cyclic olefin copolymer sheet.

A tenth aspect is a method of the ninth aspect, wherein the first composition comprises methylene chloride or tetrahydrofuran.

An eleventh aspect is a method of any of the first five aspects, wherein the thermoplastic polymeric sheet is a styrene maleic anhydride sheet.

A twelfth aspect is a method of any of the first eleven aspects, wherein the second composition comprises water or a C1-C4 unsubstituted alcohol.

A thirteenth aspect is a method of any of the first twelve aspects, wherein contacting the sheet having the microporous surface with the second composition comprises immersing the sheet having the microporous surface in the second composition.

A fourteenth aspect is a method of any of the first thirteen aspects, wherein the sheet having the microporous surface is contacted with the second composition before the sheet having the microporous surface begins to substantially distort or wrinkle due to evaporation of the first composition, or solvent components thereof, from the sheet.

A fifteenth aspect is a method of any of the first fourteen aspects, wherein contacting the thermoplastic polymeric sheet having the birefringence of greater than 0.0001 with the first composition comprises immersing the sheet in the first composition.

A sixteenth aspect is a method of the fifteenth aspect, further comprising removing the sheet having the microporous surface from the first composition, and wherein the sheet having the microporous surface is contacted with the second composition before the sheet having the microporous surface begins to substantially distort or wrinkle due to evaporation of the first composition, or solvent components thereof, from the sheet.

A seventeenth aspect is a method of the fifteenth aspect, further comprising removing the sheet having the microporous surface from the first composition, and wherein the sheet having the microporous surface is contacted with the second composition within 10 seconds of removing the sheet from the first composition.

An eighteenth aspect is a method of any of aspects 1-17, wherein the thermoplastic polymeric sheet has a birefringence of 0.001 or greater.

A nineteenth aspect is a method for making an article for culturing cells comprising disposing a substantially flat microporous thermoplastic polymeric sheet manufactured according to the method of any of the first eighteen aspects, or a portion thereof, in a container for cell culture.

A twentieth aspect is a method of the nineteenth aspect, wherein the container for cell culture is a multi-well plate and wherein disposing a substantially flat microporous thermoplastic polymeric sheet, or portion thereof, in the container comprises disposing the sheet or portion thereof in a well of the multi-well plate.

A twenty-first aspect is a cell culture article comprising (i) an article substrate, and (ii) a substantially flat microporous thermoplastic polymeric sheet disposed on the article substrate, wherein the sheet has an average pore size of 10 micrometers to 200 micrometers.

A twenty-second aspect is an article of the twenty-first aspect, wherein the article substrate forms the bottom of a well of a multi-well plate.

In the following, non-limiting examples are presented, which describe various embodiments of the articles and methods discussed above.

### EXAMPLES

In prior disclosures, we demonstrated that an article formed from thermoplastic polymers having a birefringence of 0.0001 or greater, preferably 0.001 or greater, can be rendered microporous by contacting the article with a composition comprising a solvent or a mixture of a solvent and a miscible non-solvent for the polymer. See, e.g., U.S. patent application Ser. No. ______, entitled “Porous and Non-Porous Cell Culture Substrate”, filed on ______, and having attorney docket no. SP10-233; U.S. patent application Ser. No. ______, entitled “Flexible Microfluidic Device with Interconnected Porous Network”, filed on ______, and having attorney docket no. SP10-234; U.S. patent application Ser. No. ______, entitled “Microporous Thermoplastic Article”, filed on ______, and having attorney docket no. SP10-243; U.S. patent application Ser. No. ______, entitled “Microporous Cell Culture Substrates”, filed on ______, and having attorney docket no. SP10-244; which patent applications are hereby incorporated herein by reference in their entireties to the extent that they do not conflict with the present disclosure. The articles included thin polymeric sheets and molded polymeric articles.

Some specific examples of solvent and non-solvent mixtures that have the appropriate RED values to form microporous surfaces on molded polystyrene articles that have a minimum birefringence of 0.0001 include but are not limited to those in Table 1 below:

TABLE 1
Solvents with appropriate RED values to form microporous polystyrene

| Solvent/Non-solvent mixture | Range v/v % |
|---|---|
| Tetrahydrofuran/water | 50/50-65/35 |
| Tetrahydrofuran/isopropanol | 35/65-45/55 |
| Tetrahydrofuran/propylene carbonate | 37/63-50/50 |
| Ethylacetate/isopropanol | 60/40-70/30 |
| Toluene/dimethyl sulfoxide | 25/75-30/70 |
| Acetone/isopropanol | 70/30-80/20 |
| 1,3 Dioxolane/water | 60/40-80/20 |

### Example 1

### Hansen Solubility Parameters for Solvents that Form Microporous Surfaces on Polystyrene Articles

Hansen solubility parameters for solvent mixtures that form microporous surfaces on a molded polystyrene cell culture plate, which had a gradient of birefringence values across the surface (with a significant portion being greater than 0.001), were determined in the following manner. First, a range of known solvents and non-solvents for polystyrene were tested on the surface of a molded polystyrene cell culture plate. 50-100 microliters of each test solvent and non-solvent were pipetted onto the surface of the polystyrene at room temperature. Observations were made under a microscope to see if the solvent dissolved the surface within a 2 min time period. Once a range of solvents and non-solvents were tested (see Table 2), the Hansen parameters, δP and δH, were plotted against each other for each test solvent. This type of two dimensional plot shows one cross section of the total three dimensional polystyrene solubility sphere.

TABLE 2
Solvents and non-solvents used to determine Hansen Solubility Parameters

Solvents
1,1,1-Trichloroethane
Methylene Dichloride
(Dichloromethane)
N-Methyl-2-Pyrrolidone
Ethyl Acetate
Dimethylformamide
n-Butyl Acetate
Chlorobenzene
Cyclohexanone
Isoamyl Acetate
1,3-Dioxolane
Toluene
Acetone
1,1-Dichloroethane
Tetrahydrofuran
Diethyl Ether
Methyl Ethyl Ketone
Non solvents
Cyclohexane
2-Propanol
Ethyl Lactate
Methanol
Dimethyl Sulfoxide
Glycerol
Water
Propylene Carbonate
1-Butanol
Ethanol

Using HSPiP software (Hansen Solubility Parameters in Practice, v.3.1) a fit of the data was calculated to determine the center coordinates of the polystyerene sphere and the solubility radius Rₒ. Data analysis using HSPiP software found the parameters to be δ_{D}=16.98, δ_{P}=6.76 and δ_{H}=4.83 with Rₒ=6.4. 50-100 micro liters of solvent/nonsolvent mixtures including tetrahydrofuran/water, tetrahydrofuran/isopropanol, tetrahydrofuran/propylene carbonate, ethylacetate/isopropanol, toluene/dimethyl sulfoxide, acetone/isopropanol, and 1,3 dioxolane/water were pipetted onto the polymer surface allowed to sit for 60 seconds then blow dried. The resulting surface features were observed under a microscope.

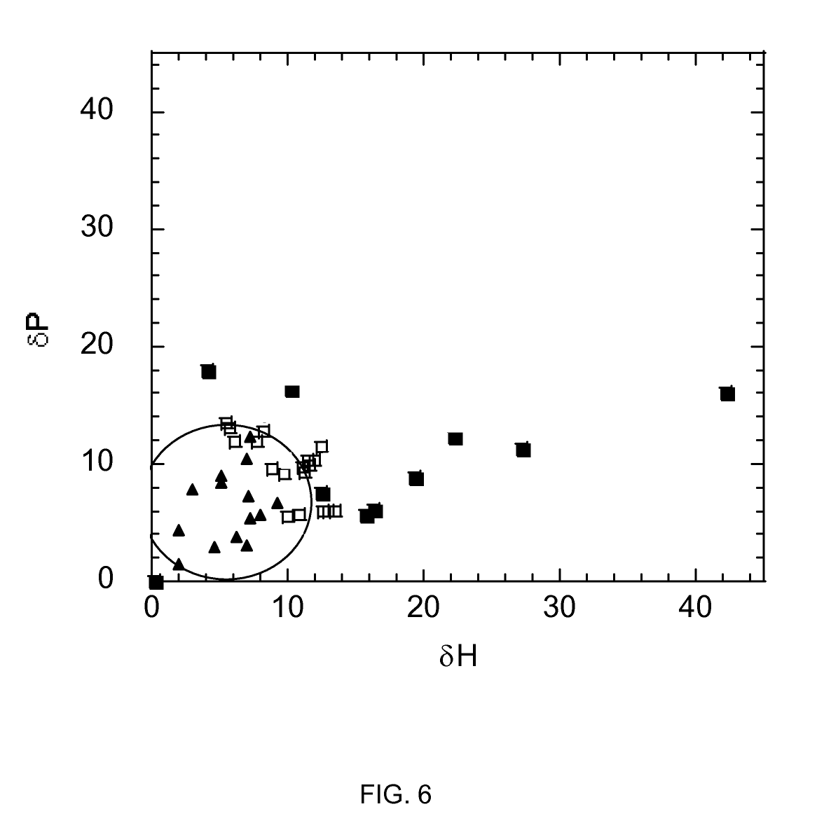

HSPiP software was used to determine the Hansen solubility parameters of the solvent/nonsolvent mixtures with the v/v % ranges shown in Table 1. The solubility parameters of the mixtures were plotted against the known solvent and non-solvent values determined earlier. FIG. 6 shows the result of the solvent, non-solvents and solvent/non-solvent mixtures that formed microporous surfaces. In FIG. 6, solid triangles represent solvents that dissolved the polymer, solid squares represent non-solvents, and open squares represent solvent/non-solvent mixtures that formed microporous surfaces. The black line circle defines the solubility boundary with radius Rₒ=6.4

The range of the solvent/non-solvent mixtures that formed microporous surfaces have RED values in the range of 0.88-1.41.

While the polymeric articles tested in this example were molded cell culture articles, the Hansen solubility parameters should be representative of polymeric sheets.

### Example 2

### Effect of Non-Solvent Bath

We have found that if thin thermoplastic sheets are rendered microporous as described in the prior provisional patent applications identified above, where the sheet is dryed following contact with the solvent (or mixture of solvent and non-solvent), the sheets tend to wrinkle or distort and do not remain flat. For many uses it is desirable for the sheets to remain flat.

As described herein, microporous thermoplastic sheets can be rendered microporous without substantial wrinkling or distortion.

In this Example, a 5 in×5 in, 3 mil (75 micron) thick film of polystyrene (Trycite 1003U, Dow) was clamped to a metal frame with 3 in×3 in opening and then immersed in a pore-forming solvent first bath of a 40/60 (v/v %) mixture of THF/isopropanol. The porous structure formed immediately after contacting the solvent. After being submerged for 75 seconds, the plasticized film was removed from the first bath and submerged in a second bath of isopropanol for 2 minutes. The rigid porous film was then removed from the isopropanol bath and blow dried with nitrogen. The dried film was flat and free of severe wrinkles or distortions. In a separate experiment, the film was removed from the first bath and blow dried with nitrogen (without being placed in the second bath).

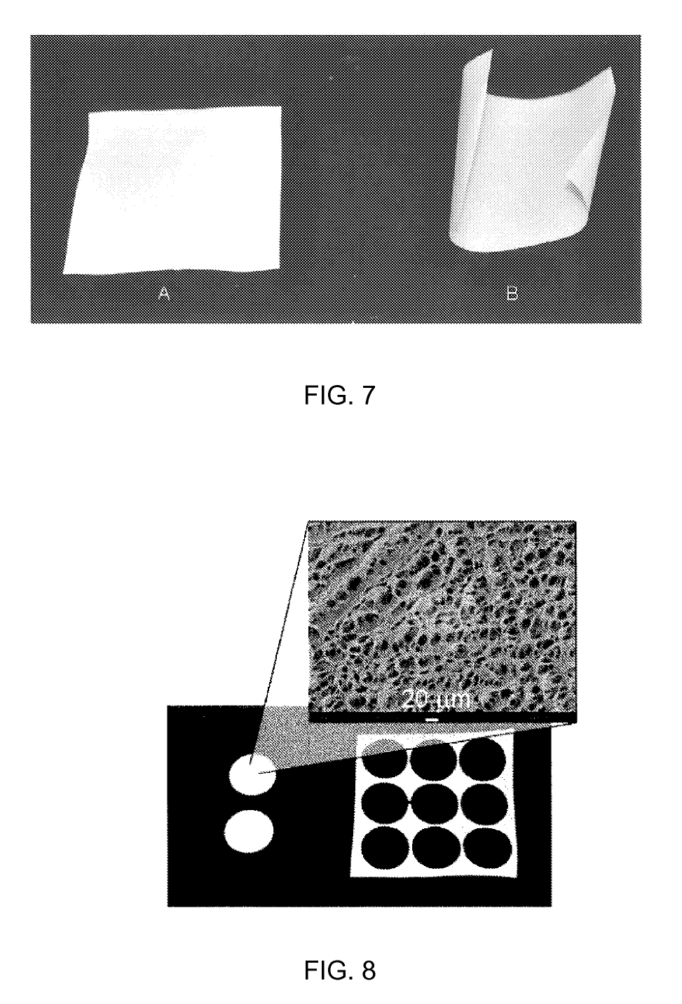

FIG. 7 shows a comparison of a film immersed in the second bath and then dried (A) to the film that was dried without immersion in the second bath (B).

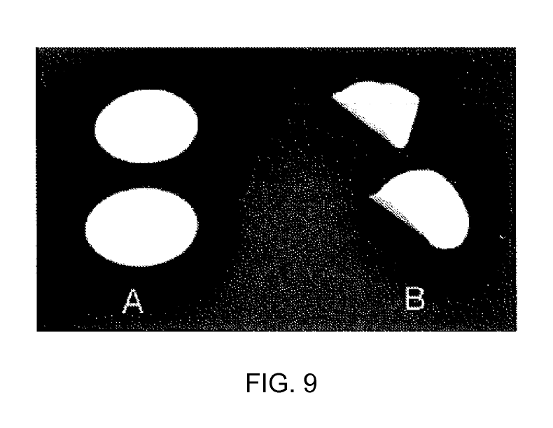

FIG. 8 shows flat microporous polystyrene film inserts for 12-well plates cut from a flat sheet of film that was treated with the two step solvent treatment process. FIG. 9 shows a comparison of flat microporous polystyrene inserts for 12-well cell culture plates where the film used to cut the inserts was immersed in the second bath and then dried (A) and where the film used to cut the inserts was dried without immersion in the second bath (B).

### Example 3

### Cell Culture on Substantially Flat Microporous Sheets

A benchmarking test was conducted to compare the function of primary hepatocytes on porous inserts made by the process described above in EXAMPLE 1 compared to collagen coated well bottom. The inserts were held on the bottom of the well with a plastic C-ring shaped clip.

Hepatocytes were cultured. After thawed, primary human hepatocytes were resuspended in MFE™ plating medium and plated at a density of 60,000/well in 96-well testing plates (collagen type I, Matrigel™ etc.). Cells were allowed to attach to the surface by incubating overnight at 37° C., 95% humidity and 5% CO₂. After hepatocytes attached to the surface, serum containing MFE™ plating medium was replaced with serum-free MFE™ culture medium. The cells were maintained for 6 days at 37° C., 95% humidity and 5% CO₂. Every 24 hours the medium was replaced with fresh medium. At the end of 6 days, the cells were used for basal gene expression analysis using quantitative real-time PCR.

### Example 4

### Gene Expression Analysis

Total RNA from primary human hepatocytes in each 96-well was isolated using the RNAqueous®-96 Kit (available from Ambion/Applied Biosystems, Austin, Tex.). The concentration of purified RNA was quantified using the Quant-iT Ribogreen RNA Reagent Kit (available from Life Technologies, Carlsbad, Calif.) according to manufacture protocol. A two-step reverse transcriptase (RT)-PCR reaction was then conducted according to TaqMan® Gene Expression Cells-to-CTT™ kit protocol (available from Life Technologies, Carlsbad, Calif.). Briefly, for each sample, 16 ng of total RNA was used to transcribe to cDNA in a 50 μl RT reaction. The entire 50 μl product of the RT reaction was then mixed with 50 μl TaqMan® Gene Expression Master Mix to make PCR reaction mixture. The entire 100-μl PCR reaction mixture was then loaded into one of 8 ports in a TaqMan® Low Density Arrays microfludic card. The TaqMan® Low Density Array was custom-made and validated by Applied Biosystems and contained probes in quadruplicate for the detection of 12 genes. PCR amplified products were detected by real-time fluorescence on an ABI PRISM 7900HT Fast Sequence Detection System (Perkin Elmer, Wellesley, Mass.).

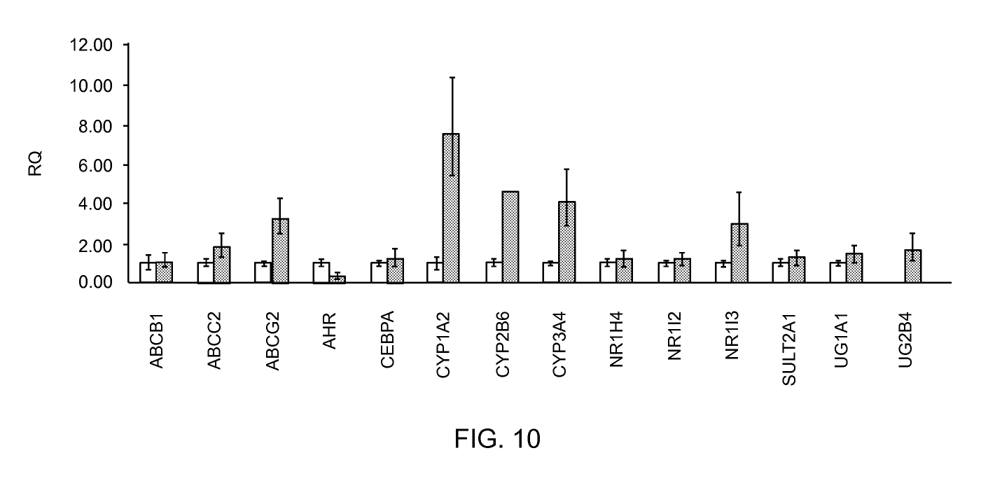

The results of the basal gene expression data for the inserts on a 12-well plate format is shown in FIG. 10, with the shaded rectangles corresponding to the cells grown on porous inserts made as described above in EXAMPLE 1 and non-shaded rectangles corresponding to collagen coated benchmark. The data indicates that cells cultured on the porous polystyrene insert made by the two-step process described herein exhibited higher functional expression of more genes than the two dimensional flat collagen-coated benchmark plate.

Thus, embodiments of MICROPOROUS THERMOPLASTIC SHEETS are disclosed. One skilled in the art will appreciate that the cell culture apparatuses and methods described herein can be practiced with embodiments other than those disclosed. The disclosed embodiments are presented for purposes of illustration and not limitation.

What is claimed is:

1. A method for fabricating a substantially flat microporous thermoplastic polymeric sheet, comprising:
providing a thermoplastic polymeric sheet having a birefringence of 0.0001 or greater and a thickness of less than 500 micrometers;
contacting the sheet with first composition having one or more solvents for the polymeric sheet, wherein the first composition has Hansen relative energy difference from the thermoplastic polymer of between 0.5 and 2 to generate a microporous surface on the sheet;
contacting the sheet having the microporous surface with a second composition that is substantially free of solvents for the sheet having the microporous surface, wherein the second composition comprises a non-solvent that is miscible with the one or more solvents in the first composition to extract the one or more solvents in the first composition from the sheet having the microporous surface; and
drying the sheet having the microporous surface to remove the second composition thereby producing the substantially flat microporous thermoplastic polymeric sheet.

2. The method of claim 1, wherein the first composition has Hansen relative energy difference from the polymer of between 0.75 and 1.6.

3. The method of claim 1, wherein the first composition comprises one or more non-solvents for the polymeric sheet.

4. The method of claim 1, wherein the thermoplastic polymeric sheet is a polystyrene sheet.

5. The method of claim 4, wherein the first composition comprises a solvent selected from the group consisting of tetrahydrofuran, ethylacetate, toluene, acetone, and 1,3 dioxolane, and a non-solvent selected from the group consisting of water, isopropanol, ethanol, propylene carbonate, and dimethyl sulfoxide.

6. The method of claim 4, wherein the first composition comprises a solvent and non-solvent mixture selected from the group consisting of (i) tetrahydrofuran and water, wherein the respective volume percentages are between 50/50 and 65/35; (ii) tetrahydrofuran and isopropanol, wherein the respective volume percentages are between 35/65 and 45/55; (iii) tetrahydrofuran and propylene carbonate, wherein the respective volume percentages are between 37/63 and 50/50; (iv) ethylacetate and isopropanol, wherein the respective volume percentages are between 60/40 and 70/30; (v) toulene and dimethyl sulfoxide, wherein the respective volume percentages are between 25/75 and 30/70; acetone and isopropanol, wherein the respective volume percentages are between 70/30 and 80/20; and (vi) 1,3 dioxolane and water, wherein the respective volume percentages are between 60/40 and 80/20.

7. The method of claim 1, wherein the thermoplastic polymeric sheet is a cyclic olefin copolymer sheet.

8. The method of claim 7, wherein the first composition comprises methylene chloride or tetrahydro furan.

9. The method of claim 1, wherein the thermoplastic polymeric sheet is a styrene maleic anhydride sheet.

10. The method of claim 1, wherein the second composition comprises water or a C1-C4 unsubstituted alcohol.

11. The method of claim 1, wherein contacting the sheet having the microporous surface with the second composition comprises immersing the sheet having the microporous surface in the second composition.

12. The method of claim 1, wherein the sheet having the microporous surface is contacted with the second composition before the sheet having the microporous surface begins to substantially distort or wrinkle due to evaporation of the first composition, or solvent components thereof, from the sheet.

13. The method of claim 1, wherein contacting the thermoplastic polymeric sheet having the birefringence of greater than 0.0001 with the first composition comprises immersing the sheet in the first composition.

14. The method of claim 13, further comprising removing the sheet having the microporous surface from the first composition, and wherein the sheet having the microporous surface is contacted with the second composition before the sheet having the microporous surface begins to substantially distort or wrinkle due to evaporation of the first composition, or solvent components thereof, from the sheet.

15. The method of claim 13, further comprising removing the sheet having the microporous surface from the first composition, and wherein the sheet having the microporous surface is contacted with the second composition within 10 seconds of removing the sheet from the first composition.

16. The method of claim 1, wherein the thermoplastic polymeric sheet has a birefringence of 0.001 or greater.

17. A method for making an article for culturing cells comprising:
disposing a substantially flat microporous thermoplastic polymeric sheet manufactured according to claim 1, or a portion thereof, in a container for cell culture.

18. The method of claim 17, wherein the container for cell culture is a multi-well plate and wherein disposing a substantially flat microporous thermoplastic polymeric sheet, or portion thereof, in the container comprises disposing the sheet or portion thereof in a well of the multi-well plate.

19. A cell culture article comprising:
an article substrate, and
a substantially flat microporous thermoplastic polymeric sheet disposed on the article substrate, wherein the sheet has an average pore size of 10 micrometers to 200 micrometers.

20. The article of claim 19, wherein the article substrate forms the bottom of a well of a multi-well plate.
